(19) United States
(12) Patent Application Publication
(10) Pub. No.: US 2011/0301460 A1
(43) Pub. Date: Dec. 8, 2011

(54) SELF-ADMINISTERED MEDICAL ULTRASONIC IMAGING SYSTEMS

(76) Inventor: Doris Nkiruka Anite, Victoria Island (NG)

(21) Appl. No.: 12/826,188

(22) Filed: Jun. 29, 2010

Related U.S. Application Data
(60) Provisional application No. 61/357,953, filed on Jun. 23, 2010.

(30) Foreign Application Priority Data
Jun. 4, 2010 (NG) NG/P/2010/374

(51) Int. Cl. A61B 8/14

(52) U.S. Cl. 600/443

(57) ABSTRACT

A medical ultrasonic transducer guiding system capable of being used by unskilled users, comprising a moveable guidance platform configured with actuators and position sensors enabling an ultrasonic transducer to be repositioned in multiple positions about a patient's body. The system may be affixed to a patient's body by vacuum or straps, and will be controlled by at least one microprocessor and associated positioning software. The device software receives transducer location information and directs the position of the ultrasonic transducer depending upon a preset algorithm, or upon direction from a remote operator. The system also has electronic circuitry to drive the ultrasonic transducer, as well as circuitry to receive the ultrasonic echo information back from the transducer, and image reconstruction hardware and software to assemble one or more images showing the interior of the patient's body. The system may be remotely operated over a network such as the Internet.

### CROSS REFERENCE TO RELATED APPLICATIONS

This application claims the priority benefit of Nigerian patent application NG/P/2010/374, entitled SELF-ADMINISTERED DIAGNOSTIC MEDICAL DEVICES AND SOFTWARE, filed on Jun. 4, 2010, and U.S. Provisional Application No. 61/357,953, filed on Jun. 23, 2010, entitled “SELF-ADMINISTERED DIAGNOSTIC MEDICAL DEVICES AND SOFTWARE”, both by Dr. Doris Nkiruka Anite inventor.

### BACKGROUND OF THE INVENTION

1. Field of the Invention

The application is in the general field of medical devices and medical diagnostics, particularly ultrasonic imaging technology.

2. Description of the Related Art

Medical imaging technology, in particular ultrasonic medical imaging technology, has revolutionized the diagnosis and care of many medical problems, such as screening for complications of pregnancy, monitoring the status of various pelvic and abdominal organs, monitoring cardiac function, and many other areas as well. Ultrasonic medical imaging technology is widely used because unlike other forms of medical imaging technology, such as X-rays, ultrasonic scans are better able to detect soft tissue problems and are also generally considered to be quite safe. Ultrasonic imaging equipment, although certainly not inexpensive, also contrasts favorably in expense to other much more expensive forms of safe medical imaging technology, such as magnetic resonance imaging (MRI) methods. Medical ultrasonic imaging is often referred to in the alternative as medical ultrasonography, diagnostic sonography, and the like.

Ultrasonic imaging technology essentially works by a sonar-like echo method. An ultrasonic transducer emits focused beams of ultrasonic energy, typically in the form of very high frequency vibrations in the 2 to 18 megahertz range. This is far above the normal range of human hearing, which generally extends no further than about 20 kilohertz, hence the name ultrasonic.

Ultrasonic imaging methods work well for imaging soft tissues, as the sound waves tend to reflect off of the interface between different soft tissues, creating an echo pattern that can be analyzed with the use of computerized equipment and translated into a visual image of the various tissues that underlie the ultrasonic transducer. The further away a particular tissue junction is from the transducer, the longer the sound wave pulse will take to travel from the transducer head to the tissue junction, and then back again to the transducer head, and this time lag thus can be used to determine the distance to that particular soft-tissue junction.

One problem with ultrasonic imaging technology, however is that this technology still requires that a skilled operator be present to move the ultrasonic transducer about the body, and interpret the images on the video monitor of the computerized ultrasonic imaging equipment. This need for a skilled operator is unfortunate, because due to advances in electronics and instrumentation, the basic costs of medical ultrasonic imaging equipment itself is likely to continue to substantially decrease over the coming years. However since skilled operators are scarce, these equipment cost improvements may not translate into broader access to this technology. Moreover, such skilled operators or workers are often not available or on call in remote locations, or outside of standard business hours, when many medical emergencies occur.

As a result, patients suffering from sudden medical problems in remote locations, or outside of standard business hours, may be denied the substantial benefits of rapid medical diagnosis that ultrasonic imaging technology might otherwise offer. As a result, the present situation is unsatisfactory. Pregnant women suffering from unexplained bleeding after normal business hours must wait until a trained ultrasonic technician can come to the scene. As a result, complications of pregnancy that might otherwise be rapidly treated can result in the loss of the fetus, the mother, or both the mother and the fetus. Many other urgent medical conditions also cannot wait until normal business hours, or the availability of an overworked technician. Thus improved methods of obtaining diagnostic medical images, in particular ultrasonic medical images, without the immediate availability of trained operators, would be of high medical utility.

### BRIEF SUMMARY OF THE INVENTION

What is needed is an automated or semi-automated device and method that can be used by local unskilled users or anyone to produce good quality medical ultrasonic images, at least in emergency situations. If such a device and/or method existed, then patients in need of urgent medical diagnosis could either use such an imaging method themselves, or with the aid of other local unskilled users, and produce useful diagnostic images. These images in turn could then either be transmitted to a remote skilled interpreter of diagnostic images, such as a remote physician or technician, by way of a conventional network such as a cell phone network, land line phone network, the Internet, radio linkages, or other telecommunications means. The remote skilled image interpreter could then examine the images and make recommendations as to what subsequent medical steps should be done.

Alternatively, even in the absence of a network link to a remote skilled interpreter, an automated or semi-automated device and method used to produce skilled medical images could still be of high utility even when used in a mode where a patient might have a scan done in a remote location or after hours setting, and the medical image either printed out or saved in a computer memory storage device for subsequent analysis by a skilled interpreter. This way the patient could, for example, come in, get a scan, and report back to a physician only if further intervention was necessary, thus saving unnecessary travel and strain on the patient.

In principle, ultrasonic medical imaging is well suited for automation and simplification or “de-skilling”. This is because, as previously discussed, the ultrasonic vibrations produced by ultrasonic transducers are generally considered to be safe. Further, with the exception that occasionally a water based gel is placed between the ultrasonic transducer and the patient's skin in order to improve the coupling between the ultrasonic transducer and the patient's skin, the process of moving the ultrasonic transducer about different regions of the patients body is not particularly complex.

Thus, according to the invention an improved device and method for producing medical diagnostic ultrasound images, suitable for unskilled users, may be obtained by constructing a device that de-skills the process of moving the ultrasonic transducer about the different regions of the patient's body. Ideally this device would also be coupled with a network based communications device that relays the ultrasonic medical images to a remote user, who may be a skilled interpreter of medical images. Alternatively or additionally, a local image storage device such as a printer or computer storage that enables the ultrasonic medical images to be read at a later time may also be coupled with the improved de-skilled ultrasonic scanner device.

There are two general ways to de-skill (simplify) the process of moving an ultrasonic transducer head or probe about a patient's body. One way is to simply automate the process, by means of one or more actuators (e.g. electric motors and the like) that move the ultrasonic transducer head in the proper manner under the control of a computer processor and software, or by a remote skilled operator, or a combination of both.

The second way is by providing a template, such as a set of interactive instructions and potentially also guidance devices to assist an unskilled user (which may be the patient or a local unskilled assistant), to properly move the ultrasonic transducer head about the body.

In both cases, the process of de-skilling or making the medical ultrasonic scanning process simpler will involve use of sensors to determine the relative position of the ultrasonic transducer on the patient's body, as well as some sort of guidance device or platform to help guide the motion of the transducer. Additionally, computational means such as at least one microprocessor, software to read the sensors and to direct the motion of the transducer will also be used. The system will also make use of the equipment normally associated with the generation and production of ultrasonic images, such as acoustic transducers, acoustic receivers, computer based processors, and the like. In principle all modes of sonography, including A-mode, B-mode, M-mode, and the various Doppler modes may run on the simplified ultrasonic guiding systems according to the invention.

Thus, in one embodiment, the invention may be viewed as a medical ultrasonic transducer guiding system capable of being used by unskilled users. This system will generally comprise at least a moveable guidance platform configured to enable an ultrasonic transducer to be repositioned in multiple positions about a patient's body. This guidance platform will usually also have or comprise sensors that detect the position of the ultrasonic transducer and produce output describing the position of the ultrasonic transducer. This transducer position sensor data will usually be processed by at least one processor (often a computer microprocessor) and associated positioning software that receives the transducer position output from the transducer location sensors. This software will direct the position of the ultrasonic transducer in a manner that may depend upon a preset algorithm, or depend on directions from a remote operator. The system will usually also have electronic circuitry to drive the ultrasonic transducer, as well as circuitry to receive the ultrasonic echo information back from the transducer. The system will additionally have image reconstruction software to take this echo information and assemble one or more images showing the interior of the patient's body.

Thus, during the course of an automated scanning session, the positioning software will direct the ultrasonic transducer to be repositioned in multiple locations about a patient's body, and the said image reconstruction software will assemble at least one image representing the interior of said patient's body. This image will be viewed by a remote skilled user, for example over a network, or on a time-shifted basis by a local skilled user at a later time.

### BRIEF DESCRIPTION OF THE DRAWINGS

FIG. 1 shows an automated medical ultrasonic transducer guiding system.

FIG. 2 shows the automated medical ultrasonic transducer guiding system from the outside.

FIG. 3 shows a detail of how the angle of the ultrasonic transducer may be altered by the actuators inside the automated medical ultrasonic transducer guiding system.

FIG. 4 shows a manually operated medical ultrasonic transducer guiding system for unskilled users.

FIG. 5 shows an example of the automated medical ultrasonic transducer guiding system, here strapped to the abdomen of a patient in a belt-like manner, and being controlled over a network, such as the internet, by a remote user.

FIG. 6 shows an example of the electronics and software used in the automated medical ultrasonic transducer guiding system.

FIG. 7 shows further details of the thin flexible membrane system and optional gel that may cover the underside of the chassis of the moveable guidance platform.

### DETAILED DESCRIPTION OF THE INVENTION

Here ultrasonic transducers will occasionally be referred to in the alternative as the ultrasonic probe, ultrasonic probe head, or occasionally the probe head or probe.

As previously discussed, the invention may be viewed as a medical ultrasonic transducer guiding system capable of being used by unskilled users. This system will generally comprise at least a moveable guidance platform configured to enable an ultrasonic transducer to be repositioned in multiple positions about a patient's body. This guidance platform will usually also have or comprise sensors that detect the position of the ultrasonic transducer and produce output describing the position of said ultrasonic transducer. This sensor data will usually be processed by at least one processor (often a computer microprocessor) and associated positioning software that receives the output from said sensors and directs the position of the ultrasonic transducer depending upon a preset algorithm, or upon further direction from a remote operator. The system will usually also have electronic circuitry to drive the ultrasonic transducer, as well as circuitry to receive the ultrasonic echo information back from the transducer. The system will additionally have image reconstruction software to take this echo information and assemble one or more images showing the interior of the patient's body. This software will generally work in conjunction with other software and electronic systems, such as conversion software to make it easier to transmit images over a network, network software, network routers, communications links, interfaces, operating systems, and the like.

During the course of an automated scanning session, the positioning software will direct the ultrasonic transducer to be repositioned in multiple locations about a patient's body, and wherein said image reconstruction software assembles at least one image representing the interior of said patient's body. This positioning software can either direct the transducer to move by issuing movement command to various mechanical actuators, such as motors, solenoids, pistons, and the like, or by issuing movement commands in an audible or written form intended to be implemented by a human user. In this later case, a processor that controls the guidance platform or other processor(s) may be hooked up to an audio output device, such as a speaker, and issue verbal commands. Alternatively the processor may be hooked up to a video display and issue written commands and/or show diagrams instructing the user as to how to use the device. Alternatively a mixture of machine transducer and human movement commands may be issued.

This moveable guidance platform will normally be affixed to a portion of the patient's body that is of interest, such as the patient's abdomen. Usually the patient will be a human patient but in some cases the patient may be an animal patient, and the device will be used for veterinary purposes. The guidance platform may be affixed by several mechanisms, including mechanical suction—for example by forming a partial vacuum in the interior of the guidance platform so that the platform sticks to the skin of a patient. Alternatively and in some cases preferably, the guidance platform may be configured in the form of a belt-like object that is strapped to the patient's body with one or more straps.

If the guidance platform does not have mechanical actuators, and is instead intended for manual use, then the guidance platform may often have at least one slot or guidance track for the ultrasonic transducer. This guidance track will allow the ultrasonic transducer to be repositioned in various different locations on the patient's body. Usually this repositioning will be done by a user, who may be an unskilled user, and in some cases may even be the patient, all in response to repositioning commands, such as verbal or video screen commands, issuing from the positioning software and processor that controls the device.

In many of the embodiments, it may be useful to connect the device to a network, such as a cell phone network, land line phone network, the internet, or other type of network so that the images produced by the ultrasonic scanner can be viewed by a remote user, who may be a skilled user such as a physician or technician. In other embodiments, this remote user may also receive ultrasonic transducer location information over the network from the guidance platform, and this remote user may in turn either transmit instructions to the processor running the guidance platform to operate mechanical actuators to move the transducer to a desired location on the patient, transmit instructions to change the parameters of the ultrasonic scan, transmit verbal instructions to local users, or some combination of the above.

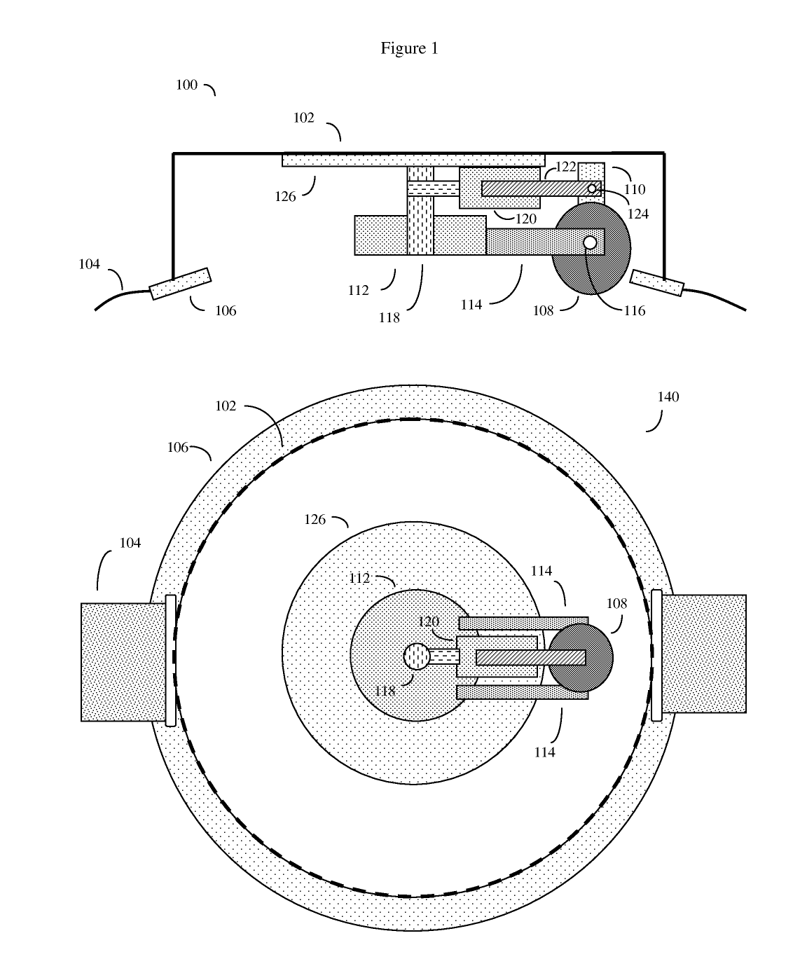

FIG. 1 shows an automated medical ultrasonic transducer guiding system. This system is based on a moveable guidance platform which is configured to enable the ultrasonic transducer to be repositioned in multiple places about a patient's body. In this particular embodiment, the platform is in the form of a portable belt that may be strapped to a patient's body. Here FIG. 1 (100) shows a cutaway side view of the platform, and FIG. 1 (140) shows a top view of the platform. The platform itself is shown as the chassis (102). The chassis, which may be rigid or semi-rigid, is held to the patient's body by way of straps (104) and a frame (106) which is generally disposed relatively flat to the patient's skin, and may be made of cushioned or pliable material for extra comfort.

The ultrasonic transducer (108) and handle (110) will often be connected to various actuators, often electronically driven actuators such as electric motors, solenoids, and the like, with appropriate gearing mechanisms. In this example, a simple rotary actuator is shown that is formed from an electric motor with rotary motion (112) affixed to the ultrasonic transducer head by a shaft (114) and joint (116). This motor (112) may in turn be affixed to the chassis by a shaft (118) or other means. If the motor (112) is affixed to the center of the chassis (102), then the motor (112) may rotate the ultrasonic transducer about a full 360 degrees of travel.

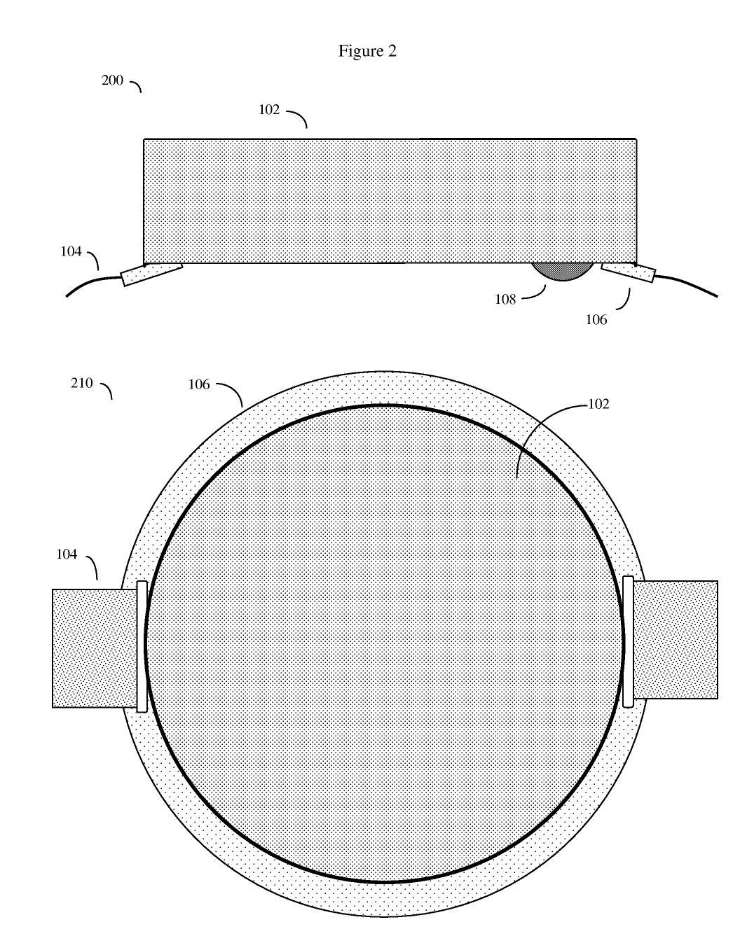

Ultrasonic transducers typically emit a relatively focused beam of ultrasonic energy, and the angle of this beam, and/or alternatively the radius of the ultrasonic transducer (108) from the center spindle or pivot (118), may be regulated by other actuator(s). In this simplified example, a second actuator (120), which may be a second electric motor, may drive a screw shaft (122) so as to either change the radius of the transducer from the shaft (118), or alternatively change the angle of the transducer (108). In this example, the screw shaft (122) is connected to the ultrasonic transducer arm (110) by a second joint (124) so that as the screw (122) is advanced or retracted by motor (120), the angle of the ultrasonic transducer head (108) will change. This will be shown in more detail in FIG. 2. These actuators (112), (120) and assorted gearing mechanisms may be affixed to the chassis (102) by a plate (126) or other mechanism.

It should be understood that although a simple rotary actuator system has been shown in this example because it is easy to draw and portray, other types of actuator systems may also be used. Other types of actuators include standard x, y actuators that may move the ultrasonic transducer in the x, y plane, as well as x, y, z actuators, x, y, z, first angle actuators, and so on.

Similarly although a simple rotary track mechanism has been drawn, again because this is simple to portray, other types of mechanisms may also be employed.

FIG. 2 shows the automated medical ultrasonic transducer guiding system from the outside. Here (200) shows the side view of a belt-like version of the transducer, showing the chassis (102), and showing the transducer (108) protruding slightly below the chassis.

Here (210) shows the top view of the belt-like version of the transducer, showing the chassis (102) which often will be encased in metal or plastic for extra protection.

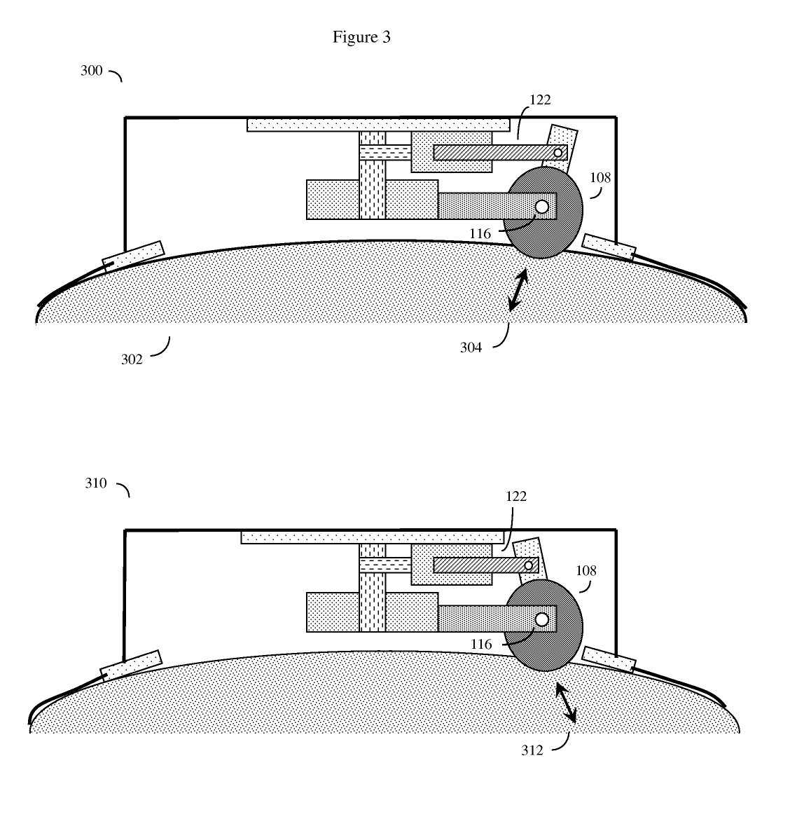

FIG. 3 shows a detail of how the angle of the ultrasonic transducer may be altered by the actuators inside the automated medical ultrasonic transducer guiding system. In FIG. 3 (300) (top), the chassis (102) of the guidance platform is affixed to a portion of a patient's body, such as the patient's abdomen (302) by way of belt straps (104). Alternatively other methods of affixing the chassis of the guidance platform, such as application of a mild vacuum, thus causing the chassis (102) to stick to the patient's body without straps, or with greater strength with straps, may also be used.

In (300), the screw shaft (122) is extended, causing the ultrasonic transducer head (108) to pivot about joint (116), causing the ultrasonic beam (304) to be directed in a first direction inward towards the center of the patient's abdomen (302).

In (310), the screw shaft (122) is retracted, causing the ultrasonic transducer head (108) to pivot in an opposite direction about joint (116), causing the ultrasonic beam (312) to be directed in a second direction outward away from the center of the patient's abdomen.

In automatic ultrasonic transducer guiding systems that use mechanical actuators, such as electric motors to move the ultrasonic transducer head, then the actuators themselves may have internal sensors that report on the relative position of the actuators to the microprocessor or other processor that the system uses to control the relative position of the actuators and thus the ultrasonic transducer head. Alternatively additional sensors, such as photoelectric sensors, electronic position sensors, and other types of sensors may be used to provide feedback to the processor and software that are used by the system to control the ultrasonic transducer position.

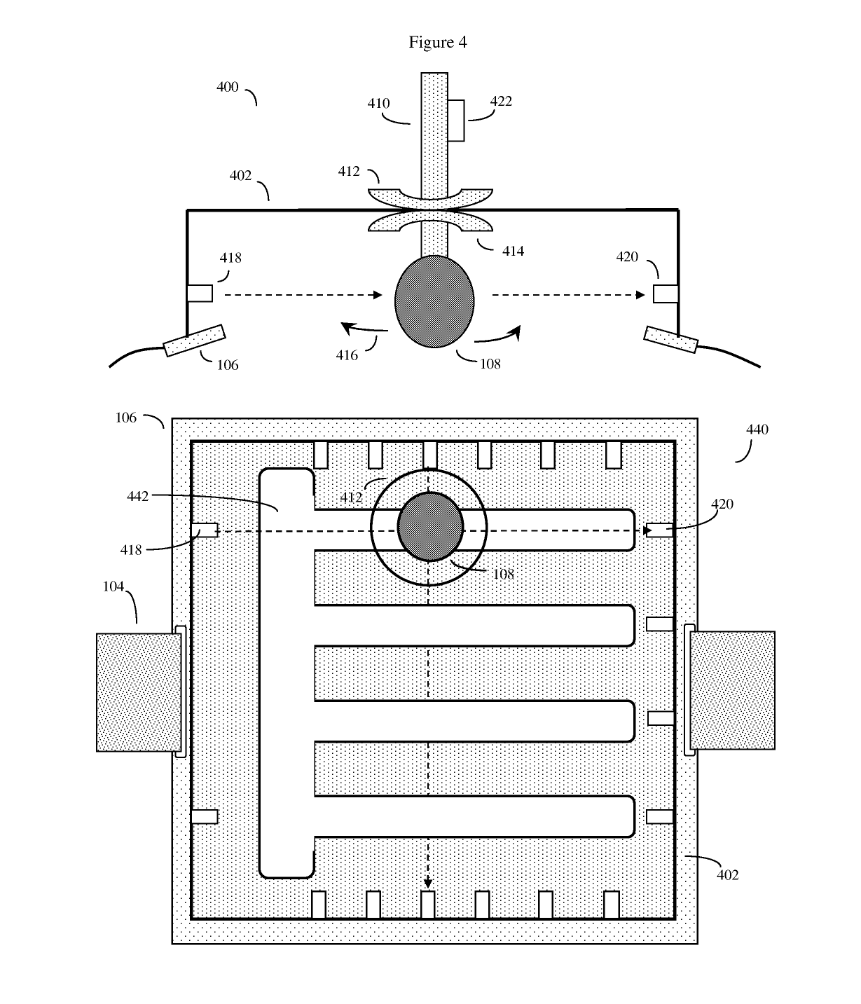

Although an automated medical ultrasonic transducer system for unskilled users is in many respects more desirable, such systems may tend to be more expensive due to the added expense of the actuators, mechanical mechanisms, and the like. In low income or low-budget situations, where expenses need to be kept to a minimum, a still simpler version of the medical ultrasonic guiding system for unskilled users is possible. This is shown in FIG. 4.

FIG. 4 shows a manually operated medical ultrasonic transducer guiding system for unskilled users. In this embodiment, the moveable guidance platform is quite different, and thus alternative nomenclature will be used. Here FIG. 4 (400) shows a cutaway side view of the platform, and FIG. 4 (440) shows a top view of the platform

In one embodiment of the manually operated system, the chassis (402) can be much more like a simple box with, for example, various slots cut into the top (442).

In the manual version of the system, the transducer handle (410) may be much longer and actually configured so that a human user can grasp onto the handle (410) and move the ultrasonic transducer head (108) through a range of locations that may, for example, be determined by the slot arrangement (442) or by other mechanical constraint mechanism. In this embodiment, the handle (410) may contain additional mechanisms such as an upper and lower curved joint region (412), (414) that hold the ultrasonic transducer (108) and handle (410) in the chassis (402), while allowing both movement through the slots (442) and even allow the ultrasonic transducer head to rock back and forth (416).

The relative position and angle of the ultrasonic transducer head (108) can be monitored by various sensors. Here optical sensors and tilt sensors are shown for simplicity. The position of the ultrasonic transducer head (108) relative to the chassis (402) may be found by putting a plurality of focused light sources (418) and light detectors (420) in various locations about the chassis. When the ultrasonic transducer head is placed in-between a particular light source (418) and detector (420), the transducer will interrupt the beam. The electric signal reported by light detector (420) can be transmitted to the microprocessor or other processor that controls the operation of the ultrasonic transducer guiding system, and by use of appropriate software, the location of the ultrasonic transducer head can be determined. Similarly tilt sensors (422) may be mounted on the handle (410) or other location of the ultrasonic transducer. These tilt sensors can be used to report the magnitude of the tilt (416) of the ultrasonic transducer to the microprocessor or other processor that controls the operation of the ultrasonic transducer guiding system.

Again, this particular embodiment is not intended to be limiting. Many other manual configurations and constraint mechanisms will also be apparent to those skilled in the art.

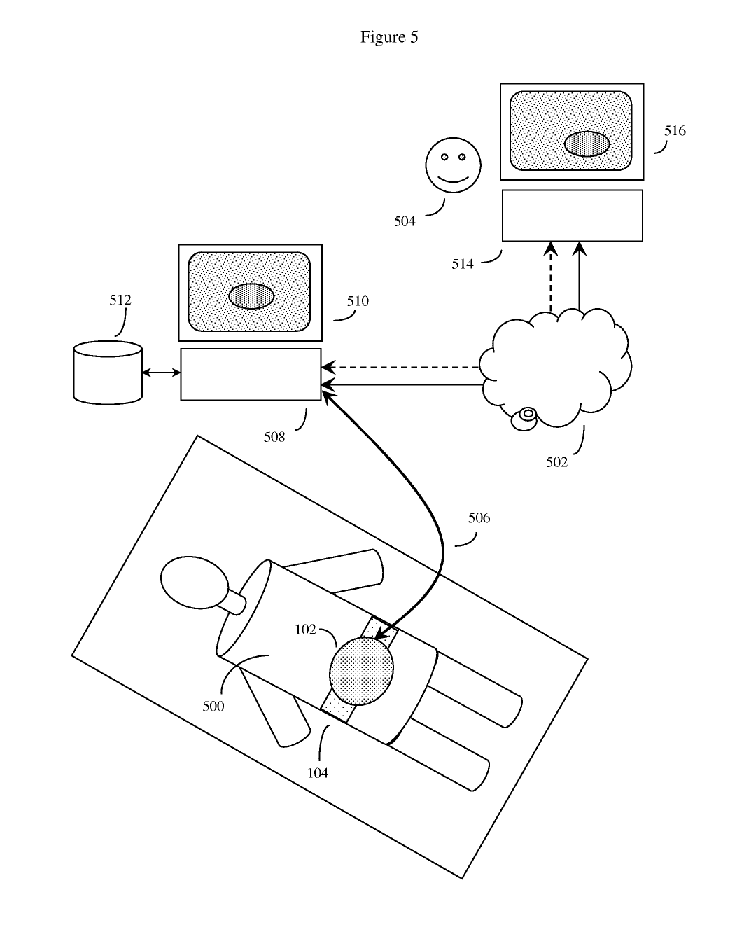

FIG. 5 shows an example of the automated medical ultrasonic transducer (102) guiding system, here strapped to the abdomen of a patient (500) in a belt-like manner (104), and being controlled over a network, such as the internet (502), by a remote user (502). In this example, the automated medical ultrasonic transducer (102) is connected by a electrical cable (506) to an electronics (508) that may, for example, contain both the processor and software that controls the automated medical ultrasonic transducer (102) as well as the electronics that drives the ultrasonic transducer head (102), and interprets the ultrasonic signal and produces an image on local display (510). The local electronics may, for example, have one or more network interfaces to networks such as cell phone networks or the internet. Additionally, the local electronics may be connected to various mechanisms for local storage and print-out of the ultrasonic results, such as a printer, disk drive, or other data storage mechanism (512).

The automated ultrasonic transducer may be controlled by more than one mechanism. In some embodiments, the local electronics (508) may take charge of controlling the movement of the ultrasonic transducer head. In this case the remote user (504) may either passively observe the images as reported over the network (502), or may even not be involved at all. Here, for example, network (502) may not even be used, and the results simply stored in local storage (512) for subsequent analysis by a more skilled user at a later time. Alternatively, remote user (504) may, for example, take command of the ultrasonic transducer head and use the remote electronics (514) send commands over the network to adjust the local electronics (508) and, for example, control the setting of the ultrasonic transducer or remotely control the scanning pattern of the ultrasonic transducer head (102). The remote user (504) may also observe the ultrasonic scan through the remote video display (516).

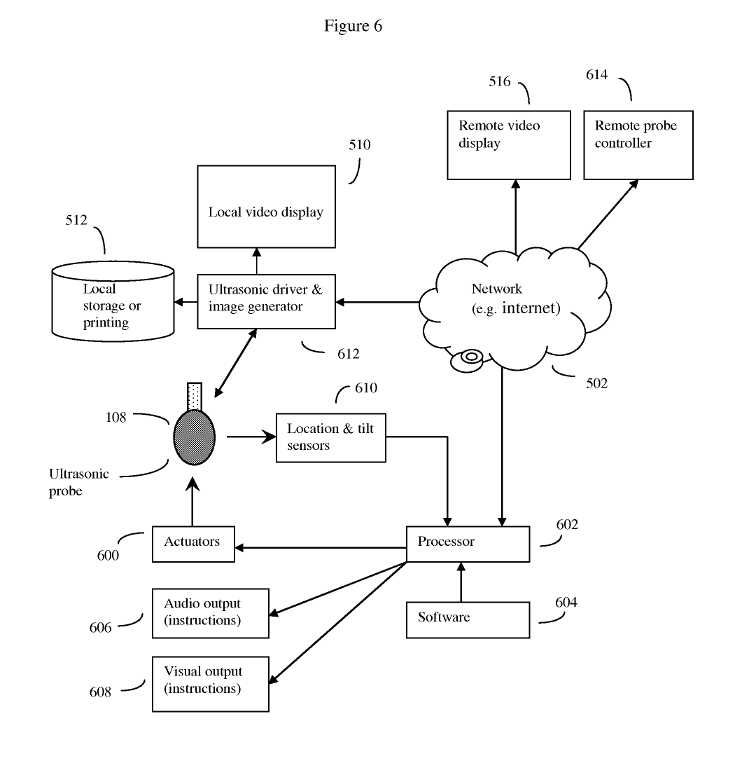

FIG. 6 shows an example of the electronics and software used in the automated medical ultrasonic transducer guiding system. Here the ultrasonic probe head (108) may have its position controlled by one or more actuators (600). These actuators may be the previously described actuators such as (112) and (120), alternative actuators, or alternatively a human patient may manually move the probe head (108) in response to instructions by the system.

The system will normally be controlled by at least one processor, such as a microprocessor (602) running under the control of software (604). In addition to running any actuators (600), the processor may also send audio commands (606) to local users (606) instructing the users as to how to move the probe, how to adjust the unit, or other commands as to when to start and stop the analysis. The processor (602) and software (604) may also send visual commands to the local user (608).

In a manual version, the location and tilt sensors (610) used to track the ultrasonic probe (108) may be the previously discussed tilt sensors (422) and/or photodetector sensors (418), (420). Alternatively other sensors may be used for either the manual or automatic versions.

In some versions, normal off-the shelf ultrasonic equipment may be retrofitted onto the transducer guiding system. In this case, the ultrasonic driver and image generator (612) may or may not be integrated in or be under the control of processor and software (602) (604). However for greater control, normally putting the ultrasonic driver and image generator (612) under control of processor (602) will generally be desirable.

As previously discussed, both processor (602) and the ultrasonic driver and image generator (612) will often have network connections allowing them to exchange data, and optionally be controlled by, remote networked equipment over a network such as the internet (502). In this case, the remote networked equipment may include a remote video display (516) and an optional electronics or software to control either the ultrasonic transducer guiding system (602), (600) and/or the ultrasonic driver and image generator (612).

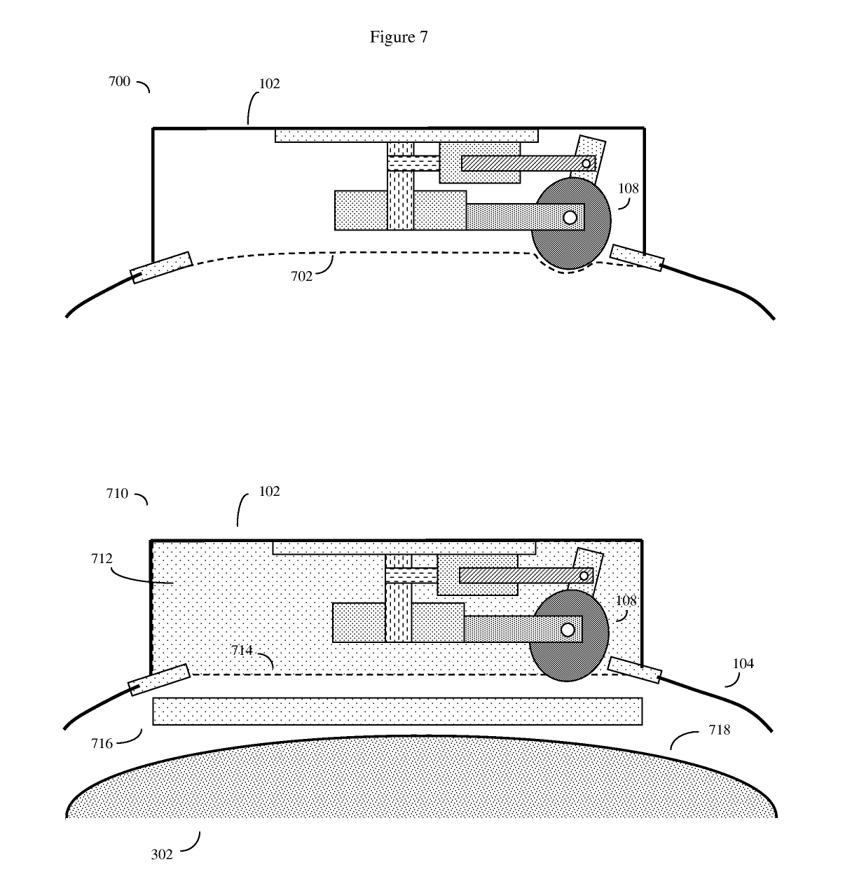

FIG. 7 shows various ways in which the ultrasonic transducer (108) and other mechanical components on the inside of the chassis (102) may be kept clean while still permitting a good ultrasonic connection between the transducer and the patient's skin. In (700), the bottom of the chassis (102) is covered by a thin flexible layer of plastic, fabric, or other membrane material. (702). This membrane material will deform around the ultrasonic transducer (108) wherever the transducer is positioned, thus maintaining a good sound conduction path between the transducer and the patient's skin. The membrane material (702) will also keep contaminants away from the potentially delicate mechanisms inside chassis (102). Ideally this membrane material will be selected to also withstand cleaning from common hospital or healthcare cleaning and disinfecting solutions such as bleach, isopropyl alcohol, soap, and other common disinfectants.

FIG. 7 (710) shows an alternative approach designed to optimize sound conduction by eliminating air pockets. Here the interior of the chassis may be filled with a fluid (712) which need not be water—other fluids such as silicon oil may also be used. The bottom of the chassis is again covered with a thin flexible membrane (714). To further insure that there are no air pockets between ultrasonic transducer head (108) and the patient's skin a layer of a water soluble gel (716), which may be provided in a pre-packaged container designed for easy application by a patient, is applied between the underside of the thin flexible membrane (714) and the patient's skin (718) before the straps (104) are tightened.

What is claimed is:

1. A medical ultrasonic transducer guiding system, comprising:
a moveable guidance platform configured to enable an ultrasonic transducer to be repositioned in multiple positions about a patient's body;
said platform additionally comprising sensors that detect the position of said ultrasonic transducer and produce output describing said position of said ultrasonic transducer;
at least one processor and positioning software that receives said output from said sensors and directs the position of said ultrasonic transducer;
said system further comprising image reconstruction software to enable echo information obtained by said ultrasonic transducer to be assembled into at least one image representing the interior of said patient's body; and
wherein said positioning software directs said ultrasonic transducer to be repositioned in multiple locations about a patient's body, and wherein said image reconstruction software assembles at least one image representing the interior of said patient's body.

2. The system of claim 1, wherein said platform is in the form of a portable belt that is strapped to the patient's body.

3. The system of claim 1, wherein said platform contains at least one guidance track for said ultrasonic transducer that enables said ultrasonic transducer to be repositioned in a plurality of different locations about said patient's body in response to repositioning commands from said positioning software.

4. The system of claim 3, wherein said positioning software issues commands to direct an unskilled user to manually reposition said ultrasonic transducer.

5. The system of claim 4, wherein said positioning software drives at least a sound output device to issue verbal commands to allow an unskilled user to manually reposition said ultrasonic transducer.

6. The system of claim 3, wherein said positioning software drives at least one mechanical actuator that automatically repositions said ultrasonic transducer.

7. The system of claim 6, wherein said at least one mechanical actuator is affixed to said platform.

8. The system of claim 1, wherein said system is electronically connected to a network, and in which said at least one image representing the interior of said patient's body may be viewed by a remote user, who may be a skilled user.

9. The system of claim 1, wherein said system is electronically connected to a network, and wherein a remote user, who may be a skilled user, interacts with said at least one processor and positioning software to direct the position of said ultrasonic transducer.

10. A medical ultrasonic transducer guiding system, comprising:
a moveable guidance platform configured to enable an ultrasonic transducer to be repositioned in multiple positions about a patient's body;
wherein said platform is in the form of a portable belt that is strapped to the patient's body;
said platform additionally comprising sensors that detect the position of said ultrasonic transducer and produce output describing said position of said ultrasonic transducer;
at least one processor and positioning software that receives said output from said sensors and directs the position of said ultrasonic transducer;
wherein said platform contains at least one guidance track for said ultrasonic transducer that enables said ultrasonic transducer to be repositioned in a plurality of different locations about said patient's body in response to repositioning commands from said positioning software;
said system further comprising image reconstruction software to enable echo information obtained by said ultrasonic transducer to be assembled into at least one image representing the interior of said patient's body;
wherein said positioning software directs said ultrasonic transducer to be repositioned in multiple locations about a patient's body, and wherein said image reconstruction software assembles at least one image representing the interior of said patient's body; and
wherein said system is electronically connected to a network, and in which said at least one image representing the interior of said patient's body may be viewed by a remote user, who may be a skilled user.

11. The system of claim 10, wherein said positioning software issues commands to direct an unskilled user to manually reposition said ultrasonic transducer.

12. The system of claim 11, wherein said positioning software drives at least a sound output device to issue verbal commands to allow an unskilled user to manually reposition said ultrasonic transducer.

13. The system of claim 11, wherein said positioning software drives at least one mechanical actuator that automatically repositions said ultrasonic transducer.

14. The system of claim 13, wherein said at least one mechanical actuator is affixed to said platform.

15. The system of claim 10, in which said system is electronically connected to a network, and wherein which a remote user, who may be a skilled user, interacts with said at least one processor and positioning software to direct the position of said ultrasonic transducer.

16. A medical ultrasonic transducer guiding system for unskilled users, comprising:
a moveable guidance platform configured to enable an ultrasonic transducer to be repositioned in multiple positions about a patient's body;
wherein said platform is in the form of a portable belt that is strapped to the patient's body;
said platform additionally comprising sensors that detect the position of said ultrasonic transducer and produce output describing said position of said ultrasonic transducer;
at least one processor and positioning software that receives said output from said sensors and directs the position of said ultrasonic transducer;
wherein said platform contains at least one guidance track for said ultrasonic transducer that enables said ultrasonic transducer to be manually repositioned in a plurality of different locations about said patient's body in response to repositioning commands from said positioning software;
wherein said positioning software drives at least a sound output device to issue verbal commands to direct said unskilled user to manually reposition said ultrasonic transducer;
said system further comprising image reconstruction software to enable echo information obtained by said ultrasonic transducer to be assembled into at least one image representing the interior of said patient's body;
wherein said positioning software directs said ultrasonic transducer to be repositioned in multiple locations about a patient's body, and wherein said image reconstruction software assembles at least one image representing the interior of said patient's body; and
wherein said system is electronically connected to a network, and wherein said at least one image representing the interior of said patient's body may be viewed by a remote user, who may be a skilled user.

17. The system of claim 16, wherein said system is electronically connected to a network, and wherein a remote user, who may be a skilled user, interacts with said at least one processor and positioning software to direct the position of said ultrasonic transducer.
